(19) United States
(12) Patent Application Publication
(10) Pub. No.: US 2005/0283216 A1
(43) Pub. Date: Dec. 22, 2005

(54) Apparatus and method for displacing tissue obstructions

(76) Inventor: Stephen T. Pyles, Ocala, FL (US)

Correspondence Address: GARDNER GROFF, P.C., 2018 POWERS FERRY ROAD, SUITE 800, ATLANTA, GA, 30339, US

(21) Appl. No.: 11/158,309

(22) Filed: Jun. 21, 2005

Related U.S. Application Data
(60) Provisional application No. 60/581,531, filed on Jun. 21, 2004.
(60) Provisional application No. 60/640,648, filed on Dec. 30, 2004.

(51) Int. Cl. A61N 1/00

(52) U.S. Cl. 607/117; 604/96.01

(57) ABSTRACT

A catheter for displacing tissue obstructions having first and second conduits extending therethrough. Preferably, the catheter has a distensible balloon positioned at its distal end in fluid communication with the first conduit, and a fluid delivery port also located at its distal end for discharging fluid from the second lumen. Preferably, the catheter also includes a stylet for guiding the catheter into and through the epidural space.

### CROSS-REFERENCE TO RELATED APPLICATIONS

This application claims priority to U.S. Provisional Patent Application Ser. No. 60/581,531, filed Jun. 21, 2004, and to U.S. Provisional Patent Application Ser. No. 60/640,648, filed Dec. 30, 2004. U.S. Provisional Patent Application Ser. Nos. 60/581,531 and 60/640,648 are hereby incorporated herein by reference in their entireties for all purposes.

### TECHNICAL FIELD

The present invention relates generally to the field of surgical methods and devices, and more particularly to an apparatus and method for displacing tissue obstructions.

### BACKGROUND OF THE INVENTION

In particular, lumbar epidural adhesions often occur as a result of a person undergoing back surgery. The adhesions, or scar tissue, tend to form around nerves and nerve roots. As a result, the adhesions apply pressure on the nerves and nerve roots, which in turn causes the person to feel pain in his or her back or legs.

To remove the adhesion, a physician would typically use an MRI (i.e., magnetic resonance imaging) to locate the general area of the adhesion. Once the area of the adhesion is known, the physician then inserts a catheter up into the area of the adhesion. Next, the physician injects fluid, such as a saline mixture, into the adhesion so as to “break up” or “blow away” the adhesion. However, the use of fluids alone does not always provide sufficient results.

In another situation, the presence of adhesions, fat, veins, and connective tissue membranes interfere with the accurate placement of percutaneous leads for spinal cord stimulation, which is used to alleviate chronic pain by stimulating the central nervous system. Typically, the percutaneous leads are inserted through a needle and placed in the epidural space, in close proximity to the spinal cord, but the epidural space that surrounds the spinal cord commonly contains fat, veins, adhesions, and connective tissue membranes which interfere with, and often prevent, the accurate placement of the electrodes. Thus, navigating through fat, veins, adhesions, and connective tissue membranes makes it difficult for the practitioner to accurately place the lead.

Therefore, a need exists for an apparatus and method which would allow for greater ease in placing percutaneous electrodes in the epidural space and which could also be used to break up lumbar adhesions.

### SUMMARY OF THE INVENTION

The present invention provides a catheter for displacing tissue obstructions, such as lumbar epidural adhesions, fibrous connective tissue membranes, fats, and veins. The catheter includes first conduit and second conduits. Preferably, the catheter has a distensible balloon positioned at its distal end in fluid communication with the first conduit, and a fluid delivery port also located at its distal end for discharging fluid from the second lumen. Preferably, the catheter further includes a stylet for guiding the catheter through the epidural space.

In another aspect, the present invention provides a method for displacing tissue obstructions or lumbar adhesions including the steps of making an incision in the patient's skin at a location on the back on the patient; inserting a catheter having a distensible balloon and a fluid delivery port at a distal end thereof through the incision; and inflating and deflating the balloon when the catheter encounters a tissue obstruction. Optionally, the method further includes the step of injecting a fluid comprising saline, corticosteroid, and/or hyaluronidase into the area of the obstruction through the fluid delivery port and then inflating and deflating the balloon to break up the adhesion.

In yet another aspect, the present invention provides a kit for performing percutaneous lysis of lumbar epidural adhesions. The kit includes a needle, a sterile drape, fluid couplings, a percutaneous lead, and a balloon-tipped catheter having a shape similar to that of the percutaneous lead, wherein all of the above are packaged in a single kit.

In still another aspect, the present invention provides a method of implanting a percutaneous lead in the spinal epidural space. The method includes the steps of performing percutaneous lysis of the epidural space using a catheter having a distensible balloon and a fluid delivery conduit and inserting a lead comprising at least one electrode into the epidural space along a path cleared by the catheter.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 shows a perspective view of a catheter for displacing tissue obstructions in accordance with an example embodiment of the present invention.

FIG. 2 shows a view of the distal or front end of the catheter of FIG. 1.

FIG. 3 shows a cross-sectional view of the distal end of the catheter of FIG. 1.

FIGS. 4A and 4B depict a flowchart representation of a method for performing percutaneous lysis of lumbar epidural adhesions using the catheter of FIG. 1.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

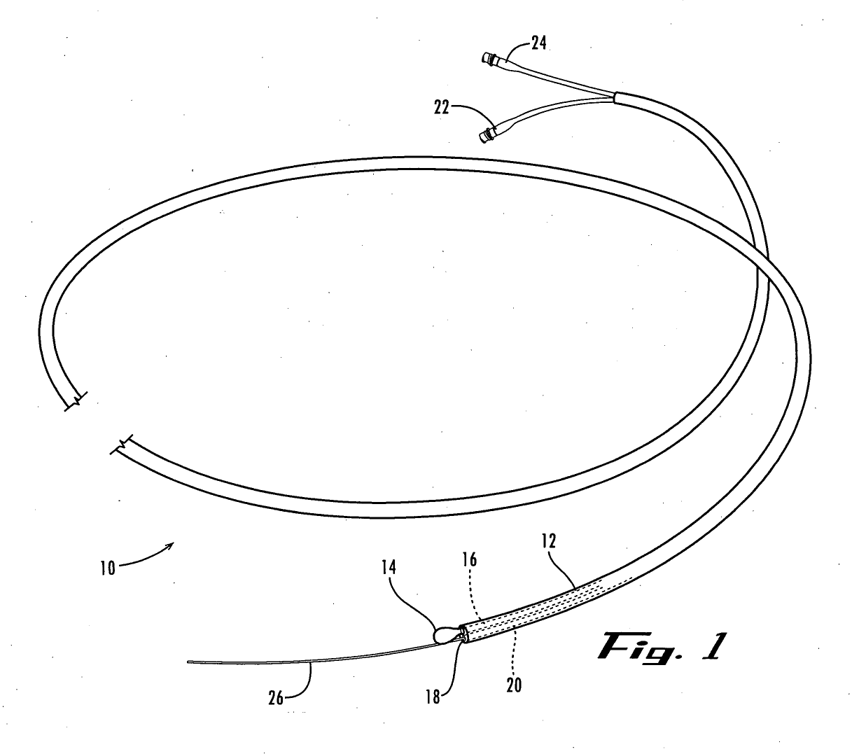

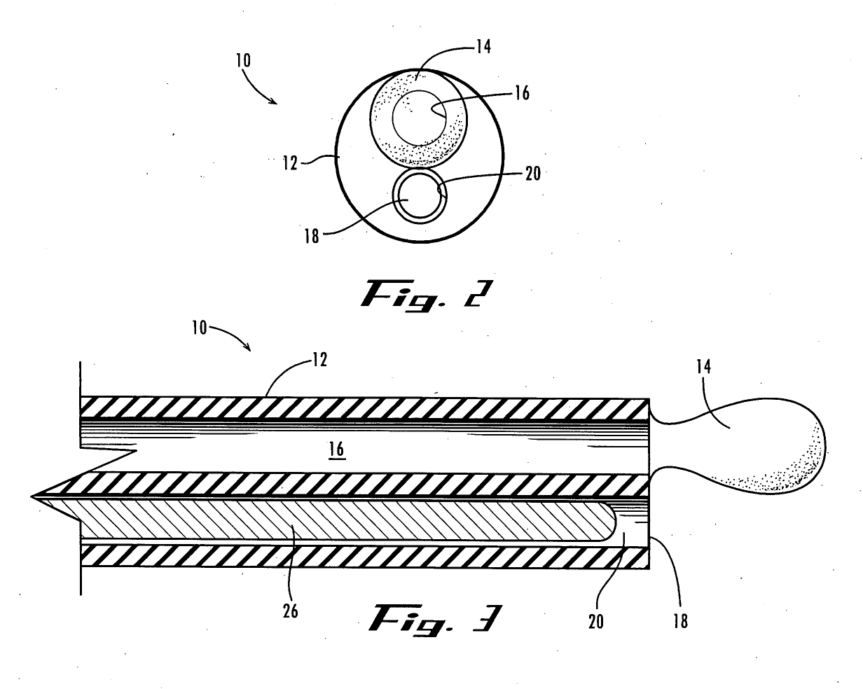

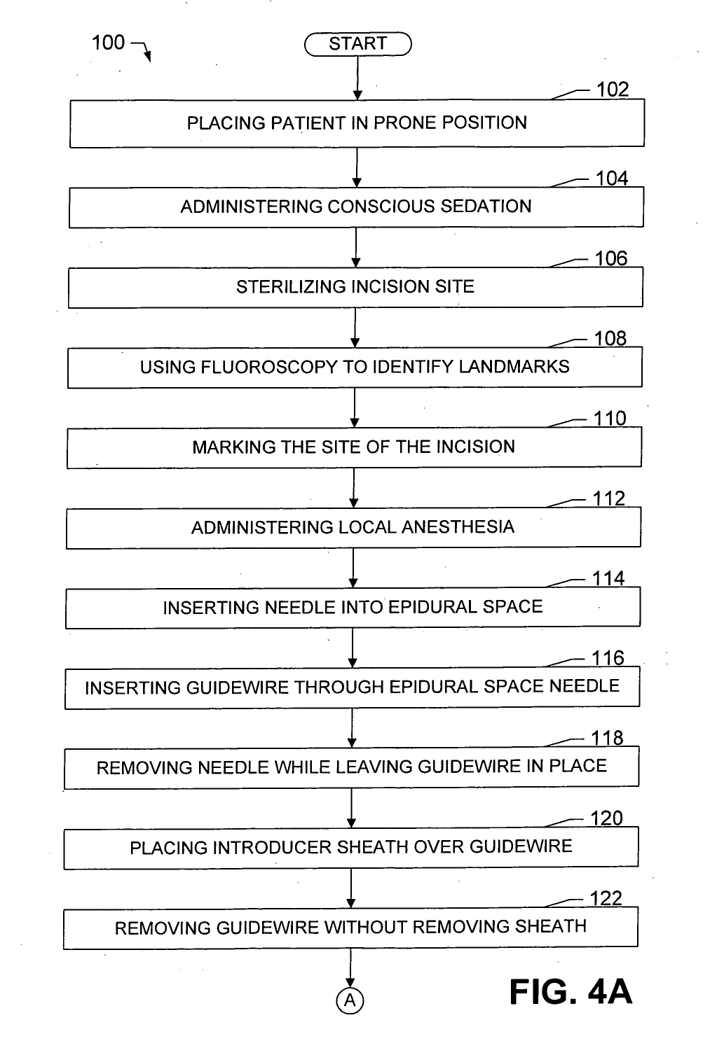

A catheter 10 for displacing tissue obstructions is described herein by way of an example embodiment shown in FIGS. 1-3. The catheter 10 has a biocompatible body 12 and a balloon 14 positioned at its distal end for displacing tissue obstructions, which can include lumbar epidural adhesions, connective tissue membranes, fats and veins. The balloon is in fluid communication with a first fluid lumen or conduit 16 extending therethrough. The first fluid lumen 16 carries a fluid, such as a sterilized liquid or air, under sufficient pressure to inflate and deflate the balloon 14. Preferably, the balloon 14 is constructed of a durable, yet distensible, material such as latex, although the present invention also contemplates the use of other distensible, biocompatible materials. The practitioner can alternately inflate and deflate the balloon 14 to displace tissues that prevent the passage or placement of the percutaneous lead.

The body 12 of the catheter 10 is preferably constructed of a biocompatible and somewhat flexible material, such as silicone, polyurethane, or polyethylene. It will be understood by those skilled in the art that various other biocompatible materials can be employed as well for the body 12.

A fluid injection port 18 is also located at the distal end of the catheter 10. The port 18 delivers fluid from a second fluid lumen or conduit 20 that extends through the body 12 of the catheter 10. The second fluid lumen 20 carries fluid for directly injecting into the area of the adhesion. For example, a mixture of saline, corticosteroid, and hyaluronidase can be injected into the site of the adhesion to reduce the inflammation via the second fluid lumen 20 and the port 18. Preferably, the volume of the mixture is not more than about 20 milliliters. Also preferably, the amount of the corticosteroid administered is limited to about 20 milligrams to no more than about 80 milligrams, and the amount of the hyaluronidase is limited to about 150 units to no more than about 1500 units.

Preferably, inlet ports of the fluid lumens 16 and 20 at the proximal end of the catheter 10 are each connected to their respective fluid sources with connectors 22 and 24, such as male “leur-lock” type connectors that couple with female leur-lock connectors of a fluid source(s), although other types of connectors for coupling the conduits of the catheter with a fluid source are within the scope of the present invention.

The catheter 10 also preferably includes a stylet 26 positioned within the second fluid conduit 20. Preferably, the stylet 26 is a slender and substantially rigid surgical wire for guiding the catheter 10 into and through the soft tissue. The stylet 26 can be straight or can be angled or curved to improve steerablilty and control. Preferably, the stylet 26 is removable from the catheter 10 such that once the catheter encounters an obstruction, the stylet can be removed and the second conduit can be fitted with a connector, such as a male leur-lock connector, and coupled to a fluid source for delivering fluid directly to the area of the obstruction. Alternatively, the stylet 26 can extend through a third lumen of the catheter 10 such that the second fluid conduit 20 can be used for fluid injection while simultaneously guiding the catheter with the stylet.

Preferably, the size and shape of the catheter 10 is substantially similar to (or slightly larger or longer than) the size and shape of a conventional and commercially available percutaneous lead so as to create a suitable path through which the percutaneous lead can be implanted. Thus, the body 12 of the catheter 10 is preferably generally cylindrical with a diameter of from about 0.6 mm to about 1.8 mm, as conventional percutaneous leads are generally cylindrical and have a diameter of about 0.8 mm to about 1.5 mm and a length of about 30 cm to about 60 cm long, or even longer. Also preferably, the catheter has a cross-sectional geometry substantially similar to that of a cross-sectional geometry of the percutaneous lead. However, it should be understood by those skilled in the art that various sizes and shapes can be employed without deviating from the scope of the present invention.

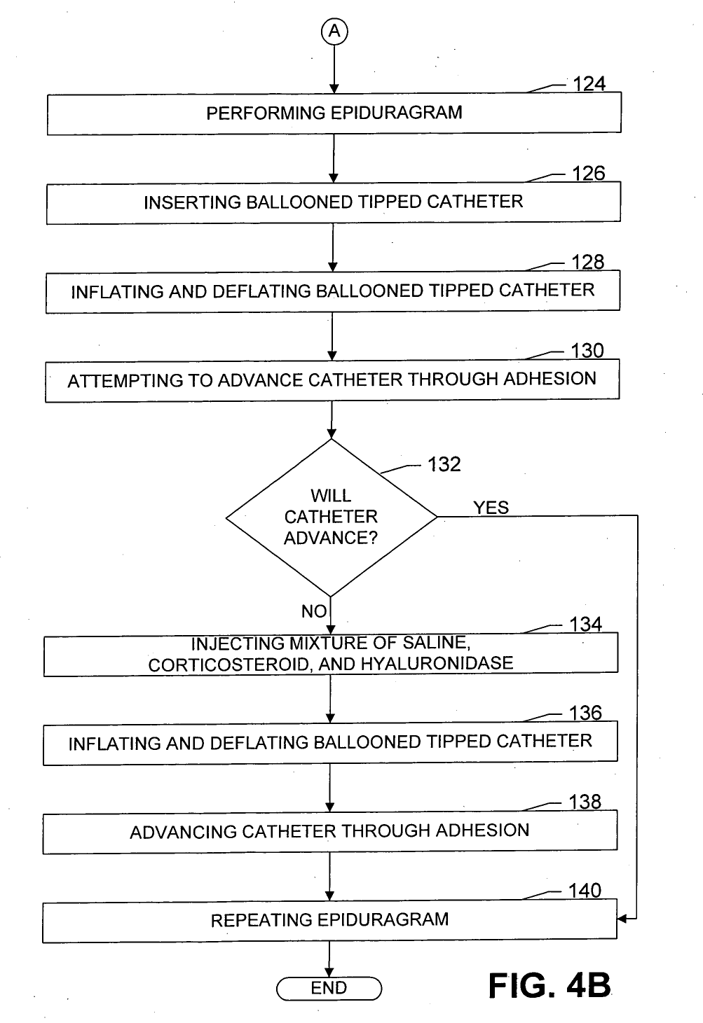

In one application, a method 100 for performing percutaneous lysis of lumbar epidural adhesions using the catheter 10 is described herein by way of an example embodiment. With reference to FIG. 4, beginning at step 102, the practitioner places the patient in a prone position, and at step 104, administers conscious sedation using for example, opioid and benzodiazepine, to the patient.

Next, at step 106, the practitioner sterilizes the area where the skin surface is to be punctured. For example, the practitioner can apply an antiseptic solution to the skin surface and then cover it with a sterile drape having an opening therein for access to the site to be punctured.

Then, at step 108, the practitioner uses fluoroscopy to identify landmarks. For example, the practitioner can use fluoroscopy to identify an oblique view of the vertebral column at L3 and the superior anterior lateral surface. At step 110, the practitioner marks the skin surface with a skin marker at the corresponding location.

At step 112, the practitioner applies a local anesthetic, such as for example 0.5% marcaine with epinephrine, to the skin and the soft tissues. Then, at step 114, the practitioner inserts a needle through the skin and into the epidural space. The practitioner can confirm the placement of the needle by injecting approximately two milliliters of a water-soluble contrast material through the needle and by viewing the resulting fluoroscopic image.

Next, the practitioner inserts a guidewire through the needle and into the epidural space at step 116. At step 118, the practitioner removes the needle from the epidural space while leaving the guidewire in the epidural space. The practitioner then places an introducer sheath over the guidewire at step 120, and removes the guidewire from the epidural space at step 122, while leaving the introducer sheath in place.

Next, the practitioner performs an epiduragram at step 124 by using approximately five milliliters of water-soluble contrast material, and preferably makes an x-ray copy of the image produced by the epiduragram.

Then at step 126, the practitioner inserts the balloon tipped catheter 10 into the sheath and advances it into the region of the lumbar epidural adhesions. Once positioned, the practitioner inflates and deflates the balloon 14 several times at step 128. After each inflation/deflation, the practitioner attempts to advance the catheter 10 through the area of the adhesion at step 130. If, at step 132, the practitioner determines that the catheter 10 will not advance, then at step 134, the practitioner injects a mixture of saline, corticosteroid, and hyaluronidase through the second conduit 20 and the port 18 and into the site of the adhesion to reduce the inflammation. Preferably, the volume of the mixture is not more than about 20 milliliters. Also preferably, the amount of the corticosteroid administered is limited to no more than about 80 milligrams, and the amount of the hyaluronidase is limited to no more than about 1500 units.

Alternatively, the practitioner can insert the catheter 10 without the use of a sheath by inserting a guidewire or stylet 26 through the second conduit 20 and using the stylet to steer the catheter into the epidural space.

After the mixture is injected, the practitioner repeats a series of inflations and deflations of the balloon 14 at step 136 and attempts to successfully advance the catheter 10 at step 138. Then, at step 140, the practitioner repeats the epiduragram with approximately five milliliters of water-soluble contrast material after the termination of the procedure. This second epiduragram documents the lysis of the adhesions by demonstrating the diminution of the filling effects.

In another application, the practitioner can use the catheter 10 to clear a path in the epidural space for placement of a percutaneous lead. In such a method, the practitioner uses the catheter 10 to perform percutaneous lysis of the epidural space, or in other words to break up the fat, veins, adhesions, and/or connective tissue membranes which would interfere with the placement of the percutaneous lead. The practitioner can inflate and deflate the balloon 14 in an effort to breach up the fat, veins, adhesions, and/or connective tissue membranes. If the practitioner cannot clear a suitable path through inflating and deflating the balloon 14, the practitioner can inject a saline solution, such as one including corticosteroid and hyaluronidase, and then re-inflate and deflate the balloon 14 to help break up the tissues. Once a suitable path has been cleared, the catheter 10 is be removed, and a percutaneous lead comprising at least one electrode, and having a size and shape generally corresponding to that of the path cleared by the catheter 10, can be inserted into the epidural space along the path cleared by the catheter and secured in place with sutures.

Optionally, the tools that the practitioner uses to insert and guide the catheter 10 into the patient can be assembled into a single kit. For example, the kit can include one or more of a needle and/or scalpel, a catheter 10, a sterile drape, fluid couplings, suturing supplies, a guidewire or stylet, a needle, an introducer sheath, and a percutaneous lead, the lead preferably generally matching the size and geometry of the catheter.

While the invention has been described with reference to preferred and example embodiments, it will be understood by those skilled in the art that a variety of modifications, additions and deletions are within the scope of the invention, as defined by the following claims.

1. A catheter for displacing tissue obstructions, comprising:
first and second conduits;
a distensible balloon positioned at a distal end of the catheter in fluid communication with the first conduit; and
a fluid delivery port located at the distal end of the catheter for discharging fluid from the second conduit.

2. The catheter of claim 1, and further comprising a stylet for guiding the catheter into and through the epidural space.

3. The catheter of claim 2, wherein the stylet is positioned within the second conduit.

4. The catheter of claim 1, wherein the first conduit carries a sterilized fluid under sufficient pressure to expand the balloon.

5. The catheter of claim 1, wherein the second conduit carries a mixture of saline, a corticosteroid, and hyaluronidase.

6. In combination, the catheter of claim 1 and a percutaneous lead, wherein the catheter has a cross-sectional geometry substantially similar to that of a cross-sectional geometry of the percutaneous lead.

7. The catheter of claim 1, wherein the balloon is formed of latex.

8. The catheter of claim 1, further comprising a leur-lock connector for coupling one of said conduits to a fluid source.

9. A method for displacing tissue obstructions, comprising:
making an incision in the patient's skin at a location on the back on the patient;
inserting a catheter having a distensible balloon and a fluid delivery port at a distal end thereof through the incision; and
inflating and deflating the balloon when the catheter encounters a tissue obstruction.

10. The method of claim 9, further comprising the step of injecting a fluid comprising saline, corticosteroid, and/or hyaluronidase into the area of the obstruction through the fluid delivery port.

11. The method of claim 10, further comprising repeating the step of inflating and deflating of the balloon after injecting the fluid.

12. The method of claim 9, further comprising the step of using fluoroscopy to guide the catheter through the epidural space.

13. The method of claim 9, further comprising placement of a percutaneous lead within a path cleared by the displacement of tissue obstructions.

14. A kit for performing percutaneous lysis of lumbar epidural adhesions, comprising:
a needle;
a sterile drape;
fluid couplings;
a percutaneous lead; and
a balloon-tipped catheter, wherein the catheter has a shape similar to that of the percutaneous lead and wherein all of the above are packaged in a single kit.

15. A method of clearing a path for implanting a percutaneous lead in the spinal epidural space, said method comprising:
performing percutaneous lysis of the epidural space using a catheter having a distensible balloon and a fluid delivery conduit.

16. The method of claim 15, further comprising inserting a lead comprising at least one electrode into the epidural space along a path cleared by the catheter.
